(19) United States
(12) Patent Application Publication
(10) Pub. No.: US 2025/0128301 A1
(43) Pub. Date: Apr. 24, 2025

(54) STERILIZATION AND DETECTION SYSTEM

(71) Applicant: Damaria Walker, Detroit, MI (US)

(72) Inventor: Damaria Walker, Detroit, MI (US)

(21) Appl. No.: 18/614,701

(22) Filed: Mar. 24, 2024

Related U.S. Application Data
(60) Provisional application No. 63/592,184, filed on Oct. 22, 2023.

(51) Int. Cl. B08B 7/02; A61L 2/025; A61L 2/10; A61L 2/26; B08B 3/02; B08B 7/04

(52) U.S. Cl. CPC B08B 7/028; A61L 2/025; A61L 2/10; A61L 2/26; B08B 3/02; B08B 7/04; A61L 2202/11; A61L 2202/14; A61L 2202/17; A61L 2202/24; B08B 2203/0211

(57) ABSTRACT

A washing system may include a washer, sprayer, emitter, sensor, and display. The washer may include a housing and one or more supports configured to receive a product associated with unwanted materials. The sprayer may be configured to remove a first quantity of unwanted materials. The emitter may be configured to remove a second quantity of unwanted materials. The sensor may be configured to detect a third quantity of unwanted materials. The display may be configured to indicate a fourth quantity of unwanted materials.

This US non-provisional patent application is based on and claims priority to U.S. provisional patent application 63/592,184 filed Oct. 22, 2023, which is hereby incorporated by reference in its entirety.

### BRIEF DESCRIPTION OF THE DRAWINGS

The following brief description of the embodiments is set forth with reference to the accompanying drawings:

FIG. 1 is a front view of an embodiment of the present disclosure, for example, a sterilization and detection system;

FIG. 2 is a front view of an embodiment of the present disclosure;

FIG. 3 is a front view of an embodiment of the present disclosure;

FIG. 4 is a front view of an embodiment of the present disclosure;

FIG. 5 is a front view of an embodiment of the present disclosure;

FIG. 6 is a front view of an embodiment of the present disclosure;

FIG. 7 is a front view of an embodiment of the present disclosure;

FIG. 8 is a front view of an embodiment of the present disclosure;

FIG. 9 is an isometric view of an embodiment of the present disclosure;

FIG. 10 is a closer view of an upper portion of an embodiment of the present disclosure;

FIG. 11 is a front view of an embodiment of the present disclosure;

FIG. 12 is a perspective view of an embodiment of the present disclosure;

FIG. 13 is a side view of an embodiment of the present disclosure;

FIG. 14 is a side view of another embodiment of the present disclosure;

FIG. 15A is a front view of an embodiment of the present disclosure;

FIG. 15B is a closer view of the embodiment of FIG. 15A;

FIG. 16A is a front view of an embodiment of the present disclosure;

FIG. 16B is a closer view of the embodiment of FIG. 16A;

FIG. 17 is a front view of an embodiment of the present disclosure;

FIG. 18 is a front view of an embodiment of the present disclosure;

FIG. 19 is a front view of an embodiment of the present disclosure; and

FIG. 20 is a closer view of the embodiment of FIG. 19.

### DETAILED DESCRIPTION

Embodiments may include systems and methods for sterilization and detection. The system may include one or more washers having a housing and one or more supports configured to receive a product associated with unwanted materials. The system may include one or more sprayers configured to remove a first quantity of unwanted materials, an emitter configured to remove a second quantity of unwanted materials, a sensor configured to detect a third quantity of unwanted materials, and a display configured to display a fourth quantity of unwanted materials.

A method of a washing system may include providing a washer having a housing and one or more supports. The method may include providing one or more sprayers, emitters, sensors, and displays operatively connected to each other. The method may include receiving by the washer a product associated with unwanted materials, spraying by the sprayer the product to remove a first quantity of unwanted materials from the product, applying energy by the emitter to remove a second quantity of unwanted materials from the product, detecting by the sensor a third quantity of unwanted materials remaining on the product, and displaying by the display a fourth quantity of unwanted materials detected on the product.

In embodiments, a washing system may comprise a washer, a sprayer, an emitter, a sensor, a display, and a wash controller. The washer may have a housing and one or more supports configured to receive a product prone to accumulation of unwanted materials. The sprayer may be configured to spray fluid to remove a first quantity of the unwanted materials. The emitter may be configured to emit energy to remove a second quantity of the unwanted materials. The sensor may be configured to detect a third quantity of the unwanted materials. The display may be configured to indicate a fourth quantity of the unwanted materials. The wash controller may operatively connect the sprayer, emitter, sensor and display.

The unwanted materials may include one or more contaminants including at least one of bioburden, pathogens, blood, body tissues, or excess fluid. The first and second quantities of the unwanted materials may include respective first and second contaminants including at least one of bioburden, pathogens, blood, body tissues, or excess fluid. The emitter may include an ultraviolet radiator configured to emit the energy, the energy may include ultraviolet light that resonates with the unwanted materials, or a combination thereof. The emitter may include an ultrasonic transducer configured to emit the energy, the energy may include vibratory energy that resonates with the unwanted materials, or a combination thereof. The sprayer and emitter may sequentially, simultaneously, or coextensively operate to remove the first and second quantities of the unwanted materials. The wash controller may activate at least one of the sprayer or emitter in response at least one the third quantity of the unwanted materials detected by the sensor, the fourth quantity of the unwanted materials indicated by the display, or a combination thereof.

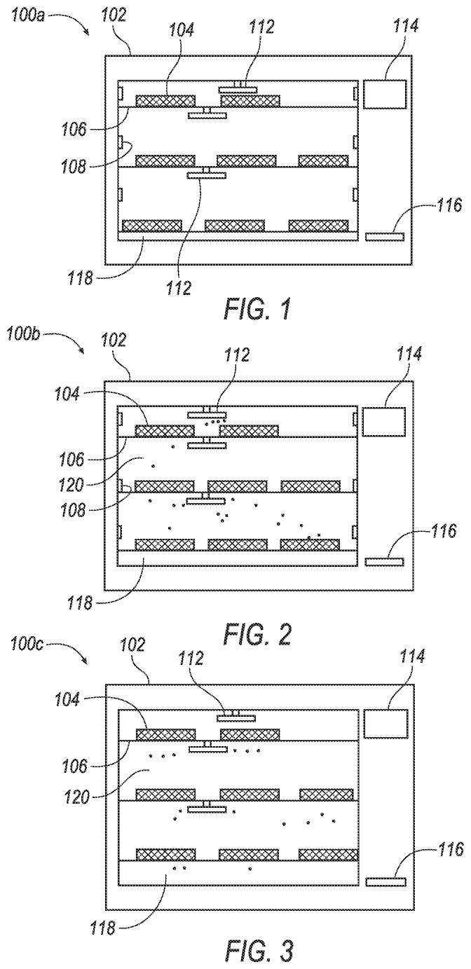

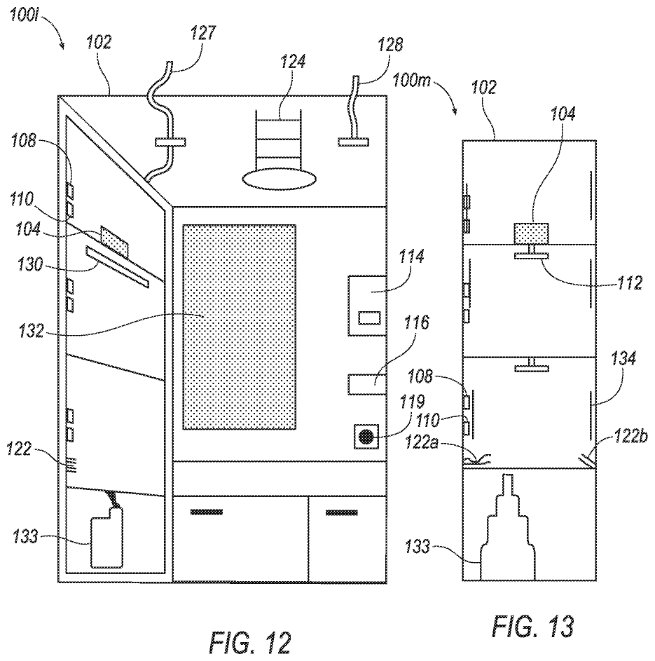

FIGS. 1-20 illustrate one or more exemplary embodiments of system 100, e.g., systems 100a-s. Any portion of the systems, methods, and processes herein may occur in any arrangement, order, or sequence. Any or all of the components and steps disclosed herein may be utilized simultaneously, others may be added, and/or others may be omitted. The drawings are provided for the purpose of illustrating exemplary embodiments and in no way limit this disclosure.

FIGS. 1-8 illustrate one or more embodiments of system 100 and methods for loading, cleaning, rinsing, draining, analysis, sampling, and/or unloading of objects 104 (e.g., tray and/or product) and associated unwanted materials. As shown in FIG. 1, system 100 may include washer 102. Washer 102 may be configured to receive one or more object 104 (e.g., a medical device or instrument). Washer 102 may include one or more middle support 106 (e.g., adjustable or fixed), sensor 108, emitter 110 (e.g., one or more ultraviolet light or “blacklight”), sprayer 112, display screen 114, test port 116, indicator 117, and base support 118 (e.g., adjustable or fixed).

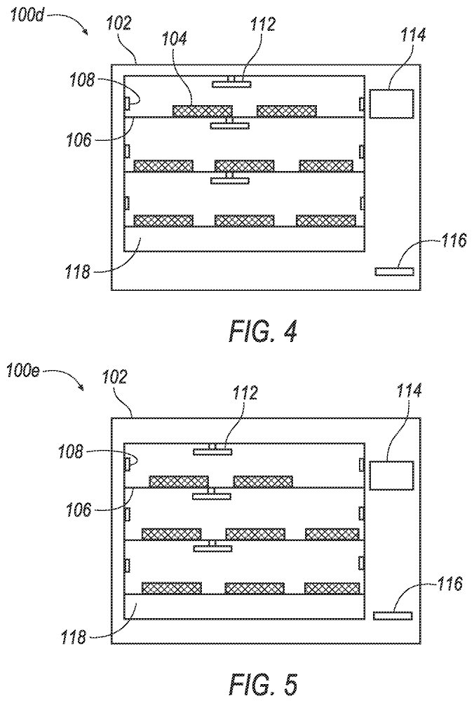

As shown in FIG. 2, washer 102 may be configured to spray a quantity (e.g., an initial or first quantity) of fluid (e.g., water, steam, and/or detergent) to clean objects 104. As shown in FIG. 3, washer 102 may be configured to spray a quantity (e.g., a further or second quantity) of the fluid to rinse objects 104 of unwanted materials. Unwanted materials may include bioburden, pathogens, blood, body tissues, excess fluid, or other harmful substances. As shown in FIG. 4, washer 102 may be configured to drain a quantity (e.g., a further or fourth quantity) of unwanted materials. As shown in FIG. 5, washer 102 may be configured to perform an analysis to detect a quantity (e.g., a further or fifth quantity) of unwanted materials.

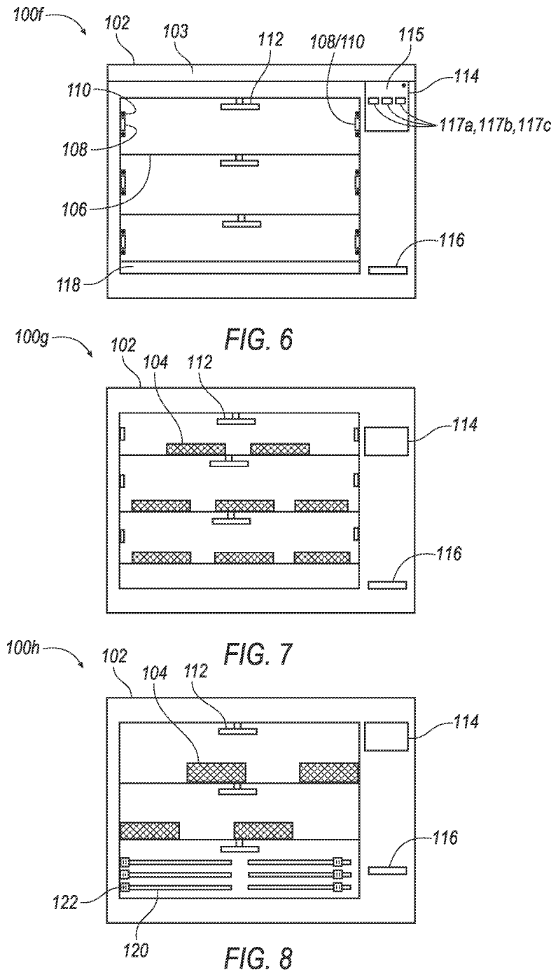

FIG. 6 illustrates an exemplary embodiment of system 100, e.g., including washer 102 configured as a detector to sample a quantity (e.g., a further or sixth quantity) of unwanted materials. System 100 may include one or more sensor 108, emitter 110 (e.g., one or more ultraviolet light), sprayer 112, display screen 114, test port 116, and/or indicator 117 operatively connected to each other to provide operations related to, e.g., detection and/or removal of unwanted materials. System 100 may include or utilize a syringe, cup, and test strips to sample the unwanted materials.

System 100 may include computer hardware including a hardware processor, physical memory, and/or hardware transmitter operatively connected to each other to provide operations regarding unwanted materials. System 100 may be configured to receive, store, display, process, compare, generate, transfer, and/or send information, e.g., regarding the unwanted materials.

As shown in FIG. 7, washer 102 may be configured to drain a quantity (e.g., a further or seventh quantity) of detected and/or unwanted materials. Washer 102 may include a door configured to selectively open for unloading and loading of objects 104.

FIG. 8 illustrates system 100 including robotics 122. Robotics 122 may include one or more robotic mechanism 120, e.g., robotic arm, robotic arm connectors, robotic arm ports, or a combination thereof. Robotics 122 may be configured to perform one or more operations on objects 104. System 100 may provide operations for robotics 122 to load, position, move, measure, and unload objects 104.

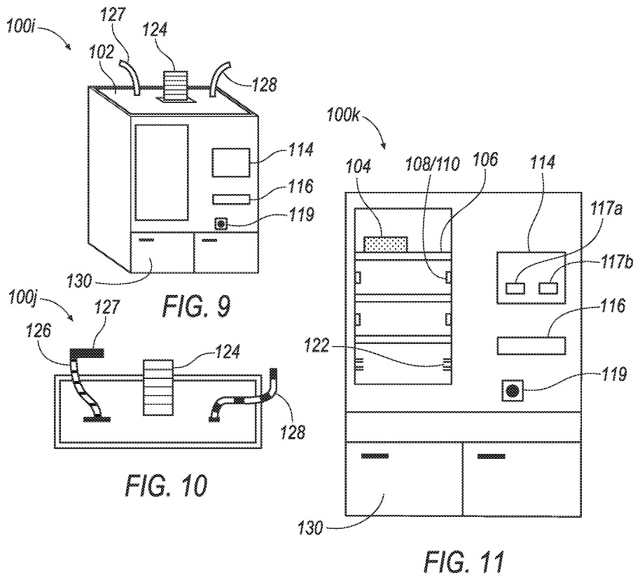

FIGS. 9-13 illustrate one or more embodiments of system 100. System 100 may include washer 102, sensor 108, emitter 110, display screen 114, tester 116, switch 119 (e.g., immediate or emergency stop), robotics 122 (e.g., robotic arm connectors 122a, 122b), duct 124 (e.g., heat and/or cooling duct to control temperature), first and second fluid lines 127, 128 (e.g., cold and/or hot fluid lines), shelf support 130, sidewall 132 (e.g., contrast background for sensor 108 and/or emitter 110), container 133 (e.g., for detergent), sidewall 132, and scanner plates 134 (e.g., for detecting unwanted materials).

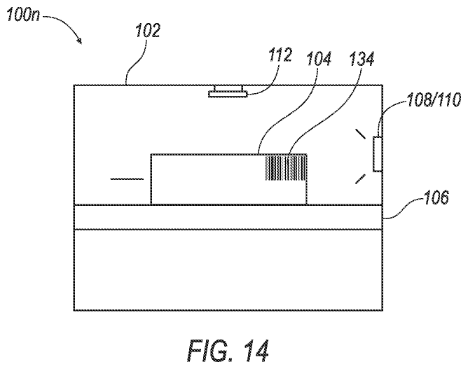

FIG. 14 illustrates system 100 configured for automated rewashing. System 100 may be configured to measure a cleanliness level of object 104 (e.g., on a tray) using sensor 108, e.g., to measure unwanted materials. Object 104 may include object identifier 134 (e.g., human readable, machine readable, or bar code) including object information. Washer 102 may be configured to apply a spray via sprayer 112 in response to the cleanliness level and object identifier 134.

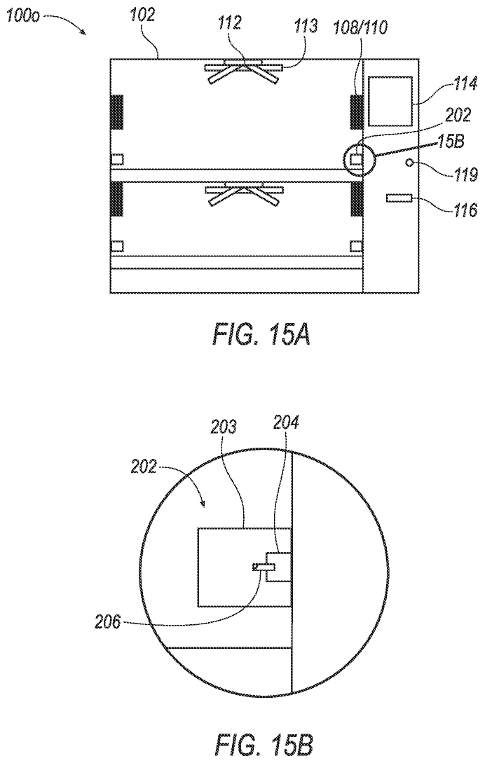

FIGS. 15A-15B illustrate system 100 configured for automated cleaning of medical instruments. System 100 may include interconnect port 202 adapted to releasably connect to and operationally engage one or more medical instruments such as robotic arms and/or cannulated instruments. Interconnect port 202 may be configured to connect to a corresponding connector of a medical instrument. As shown in FIG. 15B, interconnect port 202 may include connector housing 203, connector base 204, and controls interconnect 206. Interconnect port 202 may be integral to or removable from system 100.

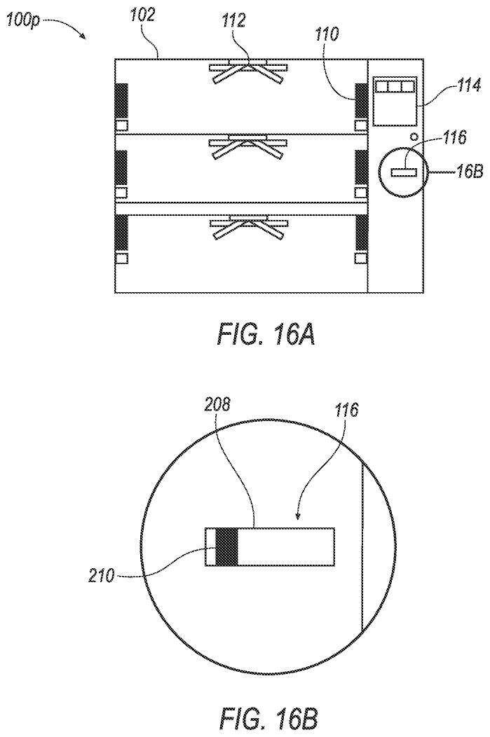

FIGS. 16A-16B illustrate system 100 configured for detection and removal of unwanted materials. Unwanted materials may include harmful materials such as anything that may be harmful to a patient during surgical procedures, e.g., infectious diseases. System 100 may include test port 116. Test port 116 may be configured to receive tester 208, e.g., to ensure the safety of a patient. As shown in FIG. 16B, tester 208 may include a test strip with indicator 210 that reacts (e.g., shows one or more lines and/or colors) to the presence of harmful materials. System 100 may be optimized to minimize usage of water, cleaners, and energy.

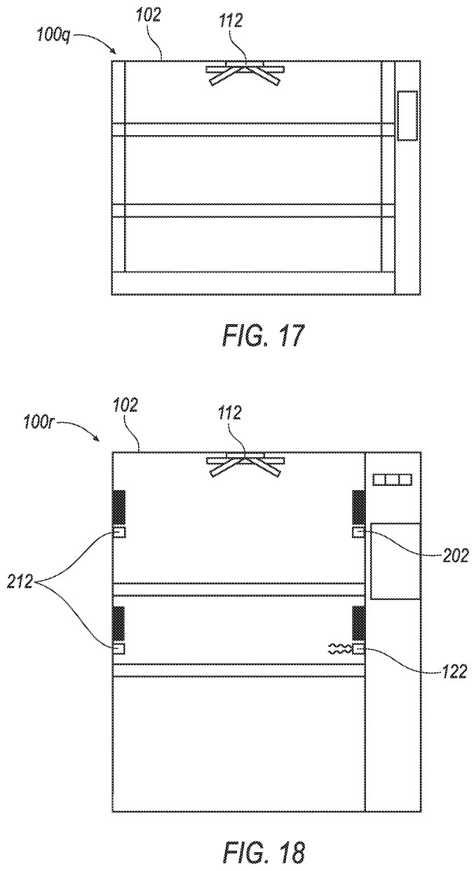

FIG. 17 may include system 100 configured for optical detection of unwanted materials. System 100 may include washer 102 having one or more emitters 210. Emitter 210 may include one or more ultraviolet lights. Emitter 210 may be configured to assist in the identification of bioburden and/or blood. Emitter 210 may be positioned on the first and/or second sides of washer 102. Emitter 210 may be activated in response to user inputs, washer malfunction, or emergency situations (e.g., catastrophes, wars, etc.). Emitter 210 may be configured to optimize usage of energy, water, and cleaners.

FIG. 18 illustrates system 100 configured for ultrasonic cleaning. System 100 may include washer 102 including jets 212. Jets 212 may include ultrasonic jets having an ultrasonic transducer that applies ultrasonic energy to a stream of fluid passing therethrough. Jets 212 may be configured to optimize removal of bioburden during the washing process. Washer 102 may include engagement connector 204.

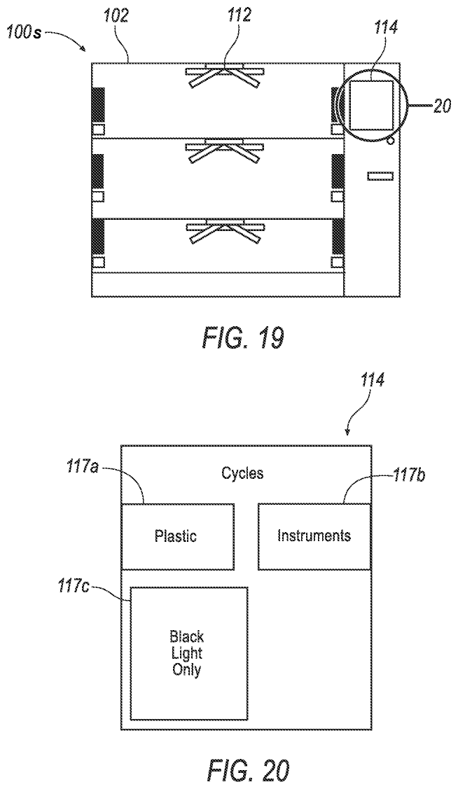

FIG. 19 may include system 100 may be configured for multi-mode cleaning. Washer 102 may include cycle controller 214, stop 216, and tester 208. As shown in FIG. 20, controller 214 may include selections for first, second, and third cycle selections 216a,b,c, e.g., for plastics, instruments, blacklight only, etc. The plastic selection 216a may utilize low temperature spray for sensitive products, e.g., medical devices or instruments. The instruments selection 216b may utilize higher temperature spray for non-sensitive products, e.g., having blood. The black light only selection 216c may be used when only a visual confirmation is needed for a product. Stop 216 may include an emergency stop for when the user needs to immediately stop washer 102 and/or open its door.

Many embodiments and applications other than the examples provided are apparent upon reading the description. The scope should be determined, not with reference to the above description, but should instead be determined with reference to the claims, along with the full scope of equivalents to which such claims are entitled. Future developments will occur in the methods discussed herein, and the disclosed systems and methods will be incorporated into such future embodiments. The embodiments of this disclosure are capable of modification, variation, and adaptation.

What is claimed is:

1. A washing system comprising:
a washer having a housing and one or more supports configured to receive a product prone to accumulation of unwanted materials;
a sprayer configured to spray fluid to remove a first quantity of the unwanted materials;
an emitter configured to emit energy to remove a second quantity of the unwanted materials;
a sensor configured to detect a third quantity of the unwanted materials;
a display configured to indicate a fourth quantity of the unwanted materials; and
a wash controller operatively connects the sprayer, emitter, sensor and display.

2. The system of claim 1, wherein the unwanted materials include one or more contaminant that includes at least one of bioburden, pathogens, blood, body tissues, or excess fluid.

3. The system of claim 1, wherein first and second quantities of the unwanted materials include respective first and second contaminants including at least one of bioburden, pathogens, blood, body tissues, or excess fluid.

4. The system of claim 1, wherein the emitter includes an ultraviolet radiator configured to emit the energy, and the energy includes ultraviolet light that resonates with the unwanted materials.

5. The system of claim 1, wherein the emitter includes an ultrasonic transducer configured to emit the energy, and the energy includes vibratory energy that resonates with the unwanted materials.

6. The system of claim 1, wherein the sprayer and emitter sequentially, simultaneously, or coextensively operate to remove the first and second quantities of the unwanted materials.

7. The system of claim 1, wherein the wash controller activates at least one of the sprayer or emitter in response at least one of:
the third quantity of the unwanted materials detected by the sensor; and
the fourth quantity of the unwanted materials indicated by the display.

8. A method of a washing system comprising:
providing a washer having a housing and one or more supports, a sprayer, an emitter, a sensor, and a display operatively connected to each other;
receiving by the washer a product associated with unwanted materials;
spraying, by the sprayer, the product to remove a first quantity of unwanted materials from the product;
applying energy by the emitter to remove a second quantity of unwanted materials from the product;
detecting by the sensor a third quantity of unwanted materials remaining on the product; and
indicating by the display a fourth quantity of unwanted materials detected on the product.

9. The method of claim 8, wherein the unwanted materials include one or more contaminant that includes at least one of bioburden, pathogens, blood, body tissues, or excess fluid.

10. The method of claim 8, wherein first and second quantities of the unwanted materials include respective first and second contaminants including at least one of bioburden, pathogens, blood, body tissues, or excess fluid.

11. The method of claim 8, wherein the emitter includes an ultraviolet radiator configured to emit the energy, and the energy includes ultraviolet light that resonates with the unwanted materials.

12. The method of claim 8, wherein the emitter includes an ultrasonic transducer configured to emit the energy, and the energy includes vibratory energy that resonates with the unwanted materials.

13. The method of claim 8, wherein the sprayer and emitter sequentially, simultaneously, or coextensively operate to remove the first and second quantities of the unwanted materials.

14. The method of claim 8, wherein the wash controller activates at least one of the sprayer or emitter in response at least one of:
the third quantity of the unwanted materials detected by the sensor; and
the fourth quantity of the unwanted materials indicated by the display.

15. A washing system comprising:
a washer having a housing and one or more supports configured to receive a product prone to accumulation of unwanted materials;
a sprayer configured to spray fluid to remove a first quantity of the unwanted materials; and
an emitter configured to emit energy to remove a second quantity of the unwanted materials.

16. The system of claim 15, wherein the unwanted materials include one or more contaminant that includes at least one of bioburden, pathogens, blood, body tissues, or excess fluid.

17. The system of claim 15, wherein first and second quantities of the unwanted materials include respective first and second contaminants including at least one of bioburden, pathogens, blood, body tissues, or excess fluid.

18. The system of claim 15, wherein the emitter includes an ultraviolet radiator configured to emit the energy, and the energy includes ultraviolet light that resonates with the unwanted materials.

19. The system of claim 15, wherein the emitter includes an ultrasonic transducer configured to emit the energy, and the energy includes vibratory energy that resonates with the unwanted materials.

20. The system of claim 15, wherein the sprayer and emitter sequentially, simultaneously, or coextensively operate to remove the first and second quantities of the unwanted materials.
